(19) United States
(12) Patent Application Publication
(10) Pub. No.: US 2010/0137261 A1
(43) Pub. Date: Jun. 3, 2010

(54) Method for treating aortic stenosis with non-antibacterial tetracycline formulations

(76) Inventors: Jani Oiva, Oulu (FI); Tuula A. Salo, Oulu (FI); Ylermi Soini, Oulu (FI); Kari Ylitalo, Oulu (FI); Antti Nissinen, Oulu (FI); Fausto Biancari, Oulu (FI); Tatu Juvonen, Oulu (FI); Jari Satta, Oulu (FI)

Correspondence Address: Ronald J Baron;Hoffmann & Baron, 6900 Jericho Turnpike, Syosset, NY, 11791, US

(21) Appl. No.: 10/592,206

(22) PCT Filed: Mar. 11, 2005
(86) PCT No.: PCT/US05/08387

§ 371 Date: Oct. 16, 2009

Related U.S. Application Data
(60) Provisional application No. 60/552,779, filed on Mar. 12, 2004.

(51) Int. Cl. A61K 31/65; A61P 9/00

(52) U.S. Cl. 514/152

(57) ABSTRACT

The present invention is for a method for treating aortic stenosis in a mammal in need thereof. The method comprises administering an effective amount of a non-antibacterial tetracycline formulation, to the mammal.

### BACKGROUND OF THE INVENTION

The aortic valve connects the heart's lower-left chamber (the ventricle) to the body's largest artery, the aorta. Aortic stenosis, also known as aortic valve stenosis, is a condition in which the aortic valve narrows. This narrowing prevents the aortic valve from opening fully, which obstructs blood flow from the heart into the aorta and to the rest of the body. As a result, the heart pumps less blood with each beat, and less blood reaches all parts of the body.

Aortic stenosis can be mild, moderate or critical. Symptoms can include fatigue, lightheadedness, chest pain or tightness, fainting, shortness of breath, heart palpitations, heart murmur or swollen ankles or feet. Left untreated, aortic stenosis can lead to serious heart problems, including heart failure and sudden death.

Treatment options for aortic stenosis include surgery and/or medications. In rare cases, the aortic valve opening can be widened using a soft, thin tube (catheter) tipped with a balloon. In this procedure (valvuloplastly) the catheter is guided through a blood vessel in the elbow or groin into the aortic valve. Once in position, the balloon is inflated, stretching the valve, then deflated and removed.

Unfortunately, valvuloplastly only provides temporary relief and the narrowing eventually returns. The risk of complications, such as suffering a stroke, is high with this procedure.

Surgical valve replacement is another surgical treatment option currently available In this procedure, the damaged aortic valve is replaced with an artificial valve. One type of valve used (mechanical) is Made of metal and synthetic materials. Another type of artificial valve used is made from animal tissue (heterograft) or human tissue (homograft).

Surgical valve replacement also involves serious risks, such as infection, and requires the patient to take blood thinners post-operation, and sometimes for the remainder of their life. There remains a number of people who are too weak or otherwise do not wish to undergo such a serious surgical procedure.

The currently available medications are prescribed only to reduce the symptoms of aortic stenosis. However, the currently available medications cannot open the narrowing or stop heart muscle problems from developing.

Therefore, the prior art treatments for aortic stenosis are limited and not without adverse effects. There is a need for a novel, alternate, and superior treatment for aortic stenosis.

The compound tetracycline is a member of a class of antibiotic compounds that is referred to as the tetracyclines, tetracycline compounds, tetracycline derivatives and the like. The compound tetracycline exhibits the following general structure:

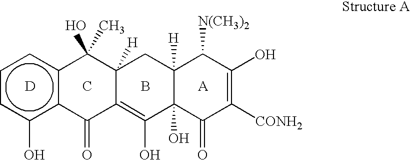

The numbering system of the tetracycline ring nucleus is as follows:

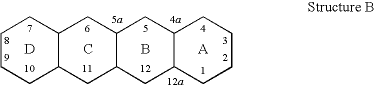

Tetracycline, as well as the terramycin and aureomycin derivatives, exist in nature, and are well known antibiotics. Natural tetracyclines may be modified without losing their antibiotic properties, although certain elements must be retained. The modifications that may and may not be made to the basic tetracycline structure have been reviewed by Mitscher in The Chemistry of Tetracyclines, Chapter 6, Marcel Dekker, Publishers, New York (1978). According to Mitscher, the substituents at positions 5-9 of the tetracycline ring system may be modified without the complete loss of antibiotic properties.

Changes to the basic ring system or replacement of the substituents at one or more of positions 4 and 10-12a, however, generally lead to synthetic tetracyclines with substantially less or effectively no antimicrobial activity. Some examples of chemically modified non-antibacterial tetracyclines (hereinafter CMTs) are 4-dedimethylaminotetracyline, 4-dedimethylaminosancycline (6-demethyl-6-deoxy-4-dedimethylaminotetracycline), 4-dedimethylaminominocycline (7-dimethylamino-6-demethyl-6-deoxy-4-dedimethylaminotetracycline), and 4-dedimethylaminodoxycycline (5-hydroxy-6-deoxy-4-dedimethylaminotetracycline).

In addition to their antimicrobial properties, tetracyclines have been described as having a number of other uses. For example, tetracyclines are also known to inhibit the activity of collagen destructive enzymes produced by mammalian (including human) cells and tissues by non-antibiotic mechanisms. Such enzymes include the matrix metalloproteinases (MMPs), including collagenases (MMP-1, MMP-8 and MMP-13), gelatinases (MMP-2 and MMP-9), and others (e.g. MMP-12, MMP-14). See Golub et al., J. Periodont. Res. 20:12-23 (1985); Golub et al. Crit. Revs. Oral Biol. Med. 2:297-322 (1991); U.S. Pat. Nos. 4,666,897; 4,704,383; 4,935,411; 4,9354,412. Also, tetracyclines have been known to inhibit wasting and protein degradation in mammalian skeletal muscle, U.S. Pat. No. 5,045,538, to inhibit inducible NO synthase, U.S. Pat. Nos. 6,043,231 and 5,523,297, and phospholipase A₂, U.S. Pat. Nos. 5,789,395 and 5,919,775, and to enhance IL-10 production in mammalian cells. These properties cause the tetracyclines to be useful in treating a number of diseases.

The object of this invention is to provide a new method for treating aortic stenosis.

### SUMMARY OF THE INVENTION

It has now been discovered that these and other objectives can be achieved by the present invention. The present invention is a method for treating aortic stenosis in a mammal in need thereof. The method comprises administering to the mammal an effective amount of a non-antibacterial tetracycline formulation.

In one embodiment, the non-antibacterial tetracycline formulation is a non-antibacterial amount of an antibacterial tetracycline. In another embodiment, the non-antibacterial tetracycline formulation is a non-antibacterial tetracycline.

### DETAILED DESCRIPTION

The invention relates to treating aortic stenosis by administering a non-antibacterial tetracycline formulation. In one embodiment of the invention, the non-antibacterial tetracycline formulation is an antibacterial tetracycline compound administered in a non-antibacterial amount, as will discussed below. For this embodiment, the tetracycline may be any such tetracycline having clinically significant antibacterial activity.

Some examples of antibacterial tetracyclines include tetracycline, as well as the 5-OH (oxytetracycline, e.g. Terramycin) and 7-Cl (chlorotetracycline, e.g. Aureomycin) derivatives, which exist in nature. Semi synthetic tetracyclines, which include, for example, doxycycline, minocycline and sancycline, can also be used for this embodiment. Examples also include demeclocycline and lymecycline.

In another embodiment of the invention, the non-antibacterial tetracycline formulation is a non-antibacterial tetracycline compound. Non-antibiotic tetracycline compounds are structurally related to the antibiotic tetracyclines, but have had their antibiotic activity substantially or completely eliminated by chemical modification, as mentioned above. For example, modified at one or more of positions 4 and 10-12a.

Non-antibiotic tetracycline compounds are preferably incapable of achieving antibiotic activity comparable to that of doxycycline unless the concentration of the non-antibiotic tetracycline is at least about ten times, and preferably at least about twenty five times, greater than that of doxycycline.

One such group of chemically modified non-antibacterial tetracyclines (CMT's) includes any of the 4-dedimethylaminotetracycline derivatives, for example, 4-dedimethylaminotetracycline (CMT-1), 6-demethyl-6-deoxy-4-de(dimethylamino)tetracycline (CMT-3), 4-dedimethylaininodoxycycline (CMT-8) and 4-dedimethylaminominocycline (CMT-10).

Some examples of suitable 4-dedimethylaminotetracycline derivatives include the following general formulae (I) through (IV):

### General Formula (I)

Structure A represents the 4-dedimethylaminosancycline (CMT-3) derivatives

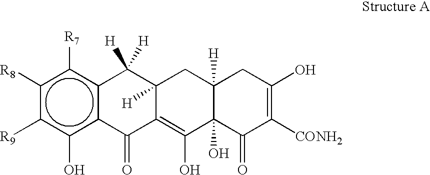

wherein R7, R8, and R9 taken together in each case, have the following meanings:

| | R7 | | R8 | R9 |
|---|---|---|---|---|
| | azido | | hydrogen | hydrogen |
| | dimethylamino | | hydrogen | azido |
| | hydrogen | | hydrogen | azido |
| | dimethylamino | | hydrogen | amino |
| | acylamino | | hydrogen | hydrogen |
| | amino | | hydrogen | nitro |
| | hydrogen | | hydrogen | (N,Ndimethyl)- |
| | | | | glycylamino |
| | amino | | hydrogen | amino |
| | hydrogen | | hydrogen | ethoxythiocarbonylthio |
| | dimethylamino | | hydrogen | acylamino |
| | dimethylamino | | hydrogen | diazonium |
| | dimethylamino | | chloro | amino |
| | hydrogen | | chloro | amino |
| | amino | | chloro | amino |
| | acylamino | | chloro | acylamino |
| | amino | | chloro | hydrogen |
| | acylamino | | chloro | hydrogen |
| | monoalkylamino | | chloro | amino |
| | nitro | | chloro | amino |
| | dimethylamino | | chloro | acylamino |
| | dimethylamino | | chloro | dimethylamino |
| | acylamino | | hydrogen | hydrogen |
| | hydrogen | | hydrogen | acylamino |
| (CMT-301) | bromo | | hydrogen | hydrogen |
| (CMT-302) | nitro | | hydrogen | hydrogen |
| (CMT-303) | hydrogen | | hydrogen | nitro |
| (CMT-304) | acetamido | | hydrogen | hydrogen |
| (CMT-305) | hydrogen | | hydrogen | acetamido |
| (CMT-306) | hydrogen | | hydrogen | dimethylamino |
| (CMT-307) | amino | | hydrogen | hydrogen |
| (CMT-308) | hydrogen | | hydrogen | amino |
| (CMT-309) | hydrogen | | hydrogen | dimethylamino- |
| | | | | acetamido |
| (CMT-310) | dimethylamino | | hydrogen | hydrogen |
| (CMT-311) | hydrogen | | hydrogen | palmitamide |
| (CMT-312) | hydrogen | hydrogen | hydrogen | CONHCH₂-pyrrolidin- |
| | | | | 1-yl |
| (CMT-313) | hydrogen | hydrogen | hydrogen | CONHCH₂-piperadin- |
| | | | | 1-yl |
| (CMT-314) | hydrogen | hydrogen | hydrogen | CONHCH₂- |
| | | | | morpholin-1-yl |
| (CMT-315) | hydrogen | hydrogen | hydrogen | CONHCH₂-piperazin- |
| | | | | 1-yl |

### General Formula (II)

Structures B through E represent the 4-dedimethylaminodoxycycline (CMT-8) derivatives

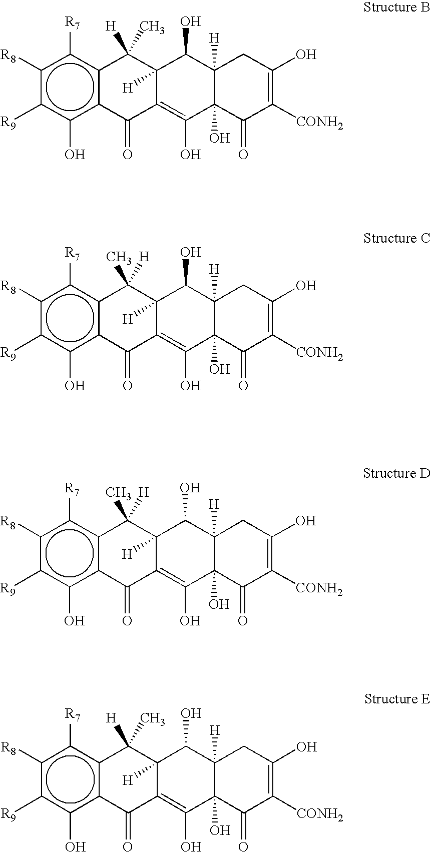

wherein R7, R8, and R9 taken together in each case, have the following meanings:

| | R7 | | R8 | R9 |
|---|---|---|---|---|
| | azido | | hydrogen | hydrogen |
| | dimethylamino | | hydrogen | azido |
| | hydrogen | | hydrogen | azido |
| | dimethylamino | | hydrogen | amino |
| | acylamino | | hydrogen | hydrogen |
| | hydrogen | | hydrogen | acylamino |
| | amino | | hydrogen | nitro |
| | hydrogen | | hydrogen | (N,N- |
| | | | | dimethyl)glycylamino |
| | amino | | hydrogen | amino |
| | hydrogen | | hydrogen | ethoxythiocarbonylthio |
| | dimethylamino | | hydrogen | acylamino |
| | hydrogen | | hydrogen | diazonium |
| | diazonium | | hydrogen | hydrogen |
| | ethoxythiocarbonylthio | | hydrogen | hydrogen |
| | dimethylamino | | chloro | amino |
| | amino | | chloro | amino |
| | acylamino | | chloro | acylamino |
| | hydrogen | | chloro | amino |
| | amino | | chloro | hydrogen |
| | acylamino | | chloro | hydrogen |
| | monoalkylamino | | chloro | amino |
| | nitro | | chloro | amino |
| (CMT-801) | hydrogen | | hydrogen | acetamido |
| (CMT-802) | hydrogen | | hydrogen | dimethyl- |
| | | | | aminoacetamido |
| (CMT-803) | hydrogen | | hydrogen | palmitamide |
| (CMT-804) | hydrogen | | hydrogen | nitro |
| (CMT-805) | hydrogen | | hydrogen | amino |
| (CMT-806) | hydrogen | | hydrogen | dimethylamino |
| | R7 | R8 | R9 | R2 |
| (CMT-807) | hydrogen | hydrogen | hydrogen | CONHCH₂-pyrrolidin- |
| | | | | 1-yl |
| (CMT-808) | hydrogen | hydrogen | hydrogen | CONHCH₂-piperadin- |
| | | | | 1-yl |
| (CMT-809) | hydrogen | hydrogen | hydrogen | CONHCH₂- |
| | | | | piperazine-1-yl |

### General Formula (III)

Structure F represents the 4-dedimethylaminominocycline (CMT-10) derivatives

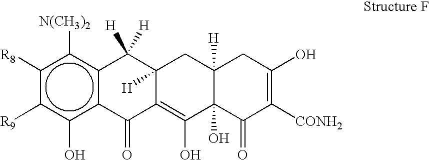

wherein R8 is hydrogen or halogen and R9 is selected from the group consisting of nitro (CMT-1002), (N,N-dimethyl)glycylamino, ethoxythiocarbonylthio. A compound related to structure F has a 7-trimethylammonium group instead of the 7-diemthylamino group, i.e. 7-trimethylammoniumsancycline (CMT-1001), and

### General Formula (IV)

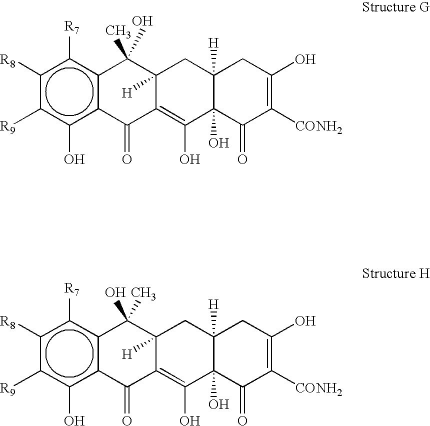

wherein R7, R8, and R9 taken together in each case, have the following meanings:

| R7 | R8 | R9 |
|---|---|---|
| amino | hydrogen | hydrogen |
| nitro | hydrogen | hydrogen |
| azido | hydrogen | hydrogen |
| dimethylamino | hydrogen | azido |
| hydrogen | hydrogen | amino |
| hydrogen | hydrogen | azido |
| hydrogen | hydrogen | nitro |
| bromo | hydrogen | hydrogen |
| dimethylamino | hydrogen | amino |
| acylamino | hydrogen | hydrogen |
| hydrogen | hydrogen | acylamino |
| amino | hydrogen | nitro |
| hydrogen | hydrogen | (N,N-dimethyl)glycylamino |
| amino | hydrogen | amino |
| diethylamino | hydrogen | hydrogen |
| hydrogen | hydrogen | ethoxythiocarbonylthio |
| dimethylamino | hydrogen | methylamino |
| dimethylamino | hydrogen | acylamino |
| dimethylamino | chloro | amino |
| amino | chloro | amino |
| acylamino | chloro | acylamino |
| hydrogen | chloro | amino |
| amino | chloro | hydrogen |
| acylamino | chloro | hydrogen |
| monoalkylamino | chloro | amino |
| nitro | chloro | amino |

Additional CMT's for purposes of the invention include 4-dedimethylaminotetracycline (CMT-1), tetracycline nitrile (CMT-2), 4-dedimethylaminochlorotetracycline (CMT-4), 4-dedimethylamino-4-hydroxytetracycline (CMT-6), 2a-dehydroxy-4-dedimethylaminotetracycline (CMT-7), and 1-deoxy-12a-dehydroxy-4-dedimethylaminotetracycline (CMT-9).

Some other examples of generic and specific tetracycline compounds that are suitable for use in the method of the invention are found in PCT/US01/16272. All such generic and specific compounds are incorporated herein by reference.

The chemically modified tetracyclines can be made by methods known in the art. See, for example, Mitscher, L. A., The Chemistry of the Tetracycline Antibiotics, Marcel Dekker, New York (1978), Ch. 6, and U.S. Pat. Nos. 4,704,383 and 5,532,227.

The invention also includes pharmaceutically acceptable salts of the above disclosed compounds. The present invention embraces salts, including acid-addition and metal salts, of the 4-dedimethylaminotetracycline compounds described herein. Such salts are formed by well known procedures. By “pharmaceutically acceptable salts” it is meant salts that do not substantially contribute to the toxicity of the compound.

Some examples of suitable salts include salts of basic tetracycline compounds and mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g. p-toluenesulfonic acids, and the like. Some examples of suitable salts of basics tetracycline compounds include bases such as sodium, potassium and ammonium hydroxide.

After preparation, the novel compounds of the present invention can be conveniently purified by standard methods known in the art. Some suitable examples include crystallization from a suitable solvent or partition-column chromatography.

The preferred pharmaceutical composition for use in the method of the invention includes a combination of the tetracycline compound in a suitable pharmaceutical carrier (vehicle) or excipient as understood by practitioners in the art. Examples of carriers and excipients include starch, milk, sugar, certain types of clay, gelatin, stearic acid or salts thereof, magnesium or calcium stearate, talc, vegetable fats or oils, gums and glycols.

The tetracycline compounds of the invention may be administered by methods known in the art, typically, systemically. Systemic administration can be enteral or parenteral. Enteral administration is a preferred route of delivery of the tetracycline, and compositions including the tetracycline compound with appropriate diluents, carriers, and the like are readily formulated. Liquid or solid (e.g., tablets, gelatin capsules) formulations can be employed.

Administration can also be accomplished by a nebulizer or liquid mist. Nebulization is a preferred route of delivery of the tetracycline in situations where the respiratory system is particularly infected. By utilizing a nebulizer, the tetracycline is taken directly into the individuals respiratory system through inspiration.

Parenteral administration of the tetracycline compounds of the invention (e.g., intravenous, intramuscular, subcutaneous injection) is also contemplated. Formulations using conventional diluents, carriers, etc. such as are known in the art can be employed to deliver the compound.

The tetracycline compound may be administered to mammals by sustained release, as is known in the art. Sustained release administration is a method of drug delivery to achieve a certain level of the drug over a particular period of time. The level typically is measured by serum concentration. For example, 40 milligrams of doxycycline may be administered by sustained release over a 24 hour period.

Further description of methods for delivering tetracycline formulations by sustained release can be found in PCT Application No. WO 02/083106, assigned to CollaGenex Pharmaceuticals, Inc. Such methods in PCT Application No. WO 02/083106 are incorporated herein by reference in its entirety.

The amount of tetracycline compound administered is any amount effective for treating aortic stenosis in the mammal. The actual preferred amounts of tetracycline compound in a specified case will vary according to the particular compositions formulated, the mode of application, and the particular subject being treated. The appropriate dose of the tetracycline compound can readily be determined by those skilled in the art.

The minimum amount of the tetracycline compound administered to a human is the lowest amount capable of providing effective treatment of the aortic stenosis. Effective treatment is partial or complete elimination of pathological characteristics associated with aortic stenosis.

The maximum amount of the tetracycline for a mammal is the highest amount that does not cause undesirable or intolerable side effects. Such doses can be readily determined by those skilled in the art.

The amount of an antibacterial tetracycline is an amount that has substantially no antibacterial activity, i.e. an amount that does not significantly prevent the growth of bacteria. For example, a tetracycline compound that has significant antibacterial activity may be administered in an amount which is 10-80% of the minimum antibacterial amount for that tetracycline compound. More preferably, the antibacterial tetracycline compound is administered in an amount which is 40-70% of the antibacterial amount.

The amount of tetracycline administered may be measured, for example, by a daily dose or by serum level. Some examples of non-antibiotic daily doses of antibiotic tetracyclines, based on steady-state pharmacokinetics, are as follows: 20 mg/twice a day for doxycycline; 38 mg of minocycline one, two, three or four times a day; 60 mg of tetracycline one, two, three or four times a day, 1000 mg/day of oxytetracycline, 600 mg/day of demeclocycline and 600 mg/day of lymecycline.

In a preferred embodiment, doxycycline is administered in a daily amount of from about 10 to about 60 milligrams, preferably 30 to 60 milligrams, but maintains a concentration in human plasma below the threshold for a significant antibiotic effect.

In an especially preferred embodiment, doxycycline hyclate is administered at a 20 milligram dose twice daily. Such a fonnulation is sold for the treatment of periodontal disease by CollaGenex Pharmaceuticals, Inc. of Newtown, Pa. under the trademark Periostat®.

Antibiotic serum levels are also known in the art. For example, a single dose of two 100 mg minocycline HCl tablets administered to an adult human results in minocycline serum levels ranging from 0.74 to 4.45 μg/ml over a period of an hour. The average level is 2.24 μg/ml.

Two hundred and fifty milligrams of tetracycline HCl administered every six hours over a twenty-four hour period produces a peak plasma concentration of approximately 3 μg/ml. Five hundred milligrams of tetracycline HCl administered every six hours over a twenty-four hour period produces a serum concentration level of 4 to 5 μg/ml.

In general, the tetracycline compound is administered in an amount which results in a serum concentration between about 0.1 and 10.0 μg/ml, more preferably between 0.3 and 5.0 μg/ml. For example, doxycycline, in a non-antibacterial formulation, is administered in an amount which results in a serum concentration between about 0.1 and 0.8 μg/ml, more preferably between 0.4 and 0.7 μg/ml.

Non-antibacterial tetracycline compounds can be used in higher amounts than antibacterial tetracyclines, while reducing or avoiding the indiscriminate killing of bacteria, and the emergence of resistant bacteria. For example, 6-demethyl-6-deoxy-4-dedimethylaminotetracycline (CMT-3) may be administered in doses of about 10 to about 200 mg/day, or in amounts that result in serum levels in humans of about 1.0 μg/ml to about 10 μg/ml. For example, a dose of about 10 to about 20 mg/day produces serum levels in humans of about 1.0 μg/ml.

For example, CMTs can be systemically administered to a mammal in a minimum amount of about 0.05 mg/kg/day to about 0.3 mg/kg/day, and a maximal amount of about 18 mg/kg/day to about 60 mg/kg/day. The practitioner is guided by skill and knowledge in the field, and the present invention includes, without limitation, dosages that are effective to achieve the desired antibacterial activity.

The tetracyclines of the present invention effectively treat aortic stenosis in a mammal in need thereof. Aortic stenosis, as discussed above, is a progressive disease resulting in the narrowing of the aortic valve.

A mammal in need of treating aortic stenosis is any mammal suffering from aortic stenosis. For example, a mammal suffering from aortic stenosis may have pathological characteristics associated with aortic stenosis such as angiogenesis (inadequate blood vessel growth) or apoptosis (programmed cell death).

A mammal which can benefit from the methods of the present invention could be any mammal. Categories of mammals include, for example, humans, farm-animals, domestic animals, laboratory animals, etc. Some examples of farm animals include cows, pigs, horses, goats, etc. Some examples of domestic animals include dogs, cats, etc. Some examples of laboratory animals include rats, mice, rabbits, guinea pigs, etc.

### Examples

The following exemplary data serves to provide further appreciation of the invention but are not meant in any way to restrict the effective scope of the invention.

A study was conducted to investigate the effectiveness of doxycycline and two, non-antibiotic, chemically modified tetracyclines (CMT-3 and CMT-8) on some pathological characteristics of aortic stenosis including matrix metalloproteinase-9 (MMP-9) synthesis, angiogenesis and apoptosis.

The study was conducted using tissue collected from four surgically removed, stenotic tricuspid valves. The valve tissue samples were incubated with various concentrations of tetracycline (TC), CMT-3, CMT-8 or solvent, for ten days.

Gelatinases (MMP-2 and MMP-9) from the valve tissue samples were assayed. The analyzed bands were related to the total protein measured using a Bio-Rad DC Protein Assay Kit (Bio-Rad, Hercules, Calif.).

All of the cultured valves expressed bands corresponding to the latent forms of MMP-2 and MMP-9. When 30 μM of CMT-3 was used, the expression of MMP-9 decreased significantly from day 6 to the end of the culture. A similar effect was also seen when 30 μM of CMT-8 was used, but only after 8 days. The 30 μM of tetracycline (TC) had an inhibitory effect on MMP-9 synthesis, but only at the end of the culture.

When compared, CMT's seemed to have a more efficient impact on MMP-9 synthesis than tetracycline. The most efficient inhibitor of MMP-9 synthesis was CMT-3.

At a concentration of 1 μM, MMP-9 inhibitory effect was seen only in CMT-3 and CMT-8 at the end of the culture. TC had no significant inhibitory effect on MMP-9 synthesis at 1 μM.

1. A method for treating aortic stenosis in a mammal in need thereof, the method comprising administering to the mammal an effective amount of a non-antibacterial tetracycline formulation.

2. The method according to claim 1, wherein the tetracycline formulation comprises a non-antibacterial amount of an antibacterial tetracycline.

3. The method according to claim 2, wherein the antibacterial tetracycline is selected from the group consisting of terramycin, aureomycin, doxycycline, minocycline, tetracycline, oxytetracycline, chlortetracycline, demeclocycline, lymecycline, or pharmaceutically acceptable salts thereof.

4. The method according to claim 1, wherein the tetracycline formulation comprises a non-antibacterial tetracycline

5. The method of claim 4, wherein the non-antibacterial tetracycline is selected from the group consisting of CMT-1, CMT-2, CMT-4, CMT-6, CMT-7, CMT-9, or CMT-10, or pharmaceutically acceptable salts thereof.

6. The method of claim 4, wherein the tetracycline is CMT-3, or its analogs, or pharmaceutically acceptable salts thereof.

7. The method according to claim 4, wherein the tetracycline is CMT-8, or its analogs, or pharmaceutically acceptable salts thereof.
